(19) United States
(12) Patent Application Publication
(10) Pub. No.: US 2017/0281392 A1
(43) Pub. Date: Oct. 5, 2017

(54) Shirt for correcting bone alignment

(71) Applicant: Toshihiro SAKAMOTO, Miyazaki (JP)

(72) Inventor: Toshihiro SAKAMOTO, Miyazaki (JP)

(21) Appl. No.: 15/231,841

(22) Filed: Aug. 9, 2016

(30) Foreign Application Priority Data
Mar. 29, 2016 (JP) 2016-064906

(51) Int. Cl. A61F 5/02; A41B 1/02

(52) U.S. Cl. CPC A61F 5/02; A41B 1/02

(57) ABSTRACT

The present invention is to provide a shirt for correcting bone alignment that has a capability to correct the alignment of the sternum and the ribs and reduce burden on user's body. The shirt for correcting bone alignment 1 includes a shirt part 10 that is formed from a stretch fabric; a first restraint part 20 that is formed on the right or the left side in the upper-half of the front body of the shirt part 10, the first restraint part 20 being less stretch than the fabric of the shirt part 10; and a second restraint part 30 that is formed on the different side from the first restraint part 20 in the upper-half of the back body of the shirt part 10, the second restraint part being less stretch than the fabric of the shirt part 10.

### TECHNICAL FIELD

This application claims priority to Japanese Patent Application No. 2016-064906 filed on Mar. 29, 2016, the entire contents of which are incorporated by reference herein.

The present invention relates to a shirt for correcting bone alignment, particularly, a shirt for correcting bone alignment that corrects the alignment of the bone in the breast of the user who is wearing.

### BACKGROUND ART

Correcting the alignment of the bone (e.g., rib) in the breast has an effect in the improvement of the posture and the relief of cervix, shoulder, and breast pains. The alignment of the bone may be improved by wearing a supporter.

As such a supporter, a wear for correcting bone alignment includes an upper supporter shaped in a vest and a lower supporter shaped in a belt has been proposed. The upper supporter has a member for spine that supports the spine; a member for right and left shoulders that supports the breast and the neighborhood of the scapulas; a member for upper right and left ribs that supports the ribs under the member for right and left shoulders; and a member for lower right and left ribs that supports the ribs under the member for upper right and left ribs. The lower supporter has an approximately inverted triangle member for sacrum that supports the neighborhood of the sacrum and a left pelvis belt and a right pelvis belt that connect with the member for sacrum (refer to Patent Document 1). This wear can appropriately correct the body by using the power to wrap the ribs and the pelvis.

### CITATION LIST

### Patent Literature

Patent Document 1: JP 2008-106386A

### SUMMARY OF INVENTION

However, the wear of Patent Document 1 restrains user's whole upper-body with the upper supporter. Therefore, the wear causes the user pain so that the user cannot use it for a long time.

An objective of the present invention is to provide a shirt for correcting bone alignment that has a capability to correct the alignment of the sternum and the ribs and reduce burden on user's body.

The first aspect of the present invention provides a shirt for correcting bone alignment that corrects the alignment of the bone in the breast of the user who is wearing, including:

a shirt part that is formed from a stretch fabric;

a first restraint part that is formed on the right or the left side in the upper-half of the front body of the shirt part, the first restraint part being less stretch than the fabric of the shirt part; and

a second restraint part that is formed on the different side from the first restraint part in the upper-half of the back body of the shirt part, the second restraint part being less stretch than the fabric of the shirt part.

According to the first aspect of the present invention, a shirt for correcting bone alignment that corrects the alignment of the bone in the breast of the user who is wearing includes a shirt part, a first restraint part, and a second restraint part. The shirt part is formed from a stretch fabric. The first restraint part is formed on the right or the left side in the upper-half of the front body of the shirt part, which is less stretch than the fabric of the shirt part. The second restraint part is formed on the different side from the first restraint part in the upper-half of the back body of the shirt part, which is less stretch than the fabric of the shirt part.

When the user wears this shirt, the first restraint part and the second restraint part which are less stretch than the fabric of the shirt part are placed opposite from each other. User's body is situated between the first restraint part and the second restraint part. The first restraint part restrains user's breast. Therefore, the shirt can correct the alignment of the bone in user's breast. Moreover, the portion other than the first restraint part and the second restraint part of the shirt part are formed from a stretch fabric so that the shirt can reduce burden on user's body. As the result, the present invention can provide a shirt for correcting bone alignment that has a capability to correct the alignment of the sternum and the ribs and reduce burden on user's body.

Some people may have the displaced bone in the right side of the breast and others the left. According to the first aspect of the present invention, for example, if the shirt part is worn inside out, the first restraint part comes to the other side in the upper-half of the front body. The second restraint part then comes to the different side from the first restraint part in the upper-half of the back body. Specifically, the first restraint part can be horizontally reversed by wearing the shirt inside out. Therefore, the first restraint part can be placed right or left depending on the alignment of the bone in user's breast.

The second aspect of the present invention provides the shirt for correcting bone alignment according to the first aspect of the present invention, further including:

a first shoulder restraint part that is placed over an area from one of the shoulder parts to the first restraint part of the shirt part, the first shoulder restraint part being less stretch than the fabric of the shirt part; and

a second shoulder restraint part that is placed over an area from the other of the shoulder parts to the second restraint part, the second shoulder restraint part being less stretch than the fabric of the shirt part.

According to the second aspect of the present invention, the shirt further includes a first shoulder restraint part and a second shoulder restraint part. The first shoulder restraint part is placed over an area from one of the shoulder parts to the first restraint part of the shirt part, which less stretch than the fabric of the shirt part. The second shoulder restraint part is placed over an area from the other of the shoulder parts to the second restraint part, which is less stretch than the fabric of the shirt part.

This shirt that the user is wearing can provide the same effect as when the user tapes the shoulder to reduce shoulder tension.

The present invention can provide a shirt for correcting bone alignment that has a capability to correct the alignment of the sternum and the ribs and reduce burden on user's body.

### BRIEF DESCRIPTION OF DRAWINGS

FIG. 1 is an in-use view of the shirt for correcting bone alignment 1 according to one embodiment of the present invention.

FIG. 2 is a front view of the shirt for correcting bone alignment 1 according to this embodiment.

FIG. 3 is a back view of the shirt for correcting bone alignment 1 according to this embodiment.

### DESCRIPTION OF EMBODIMENTS

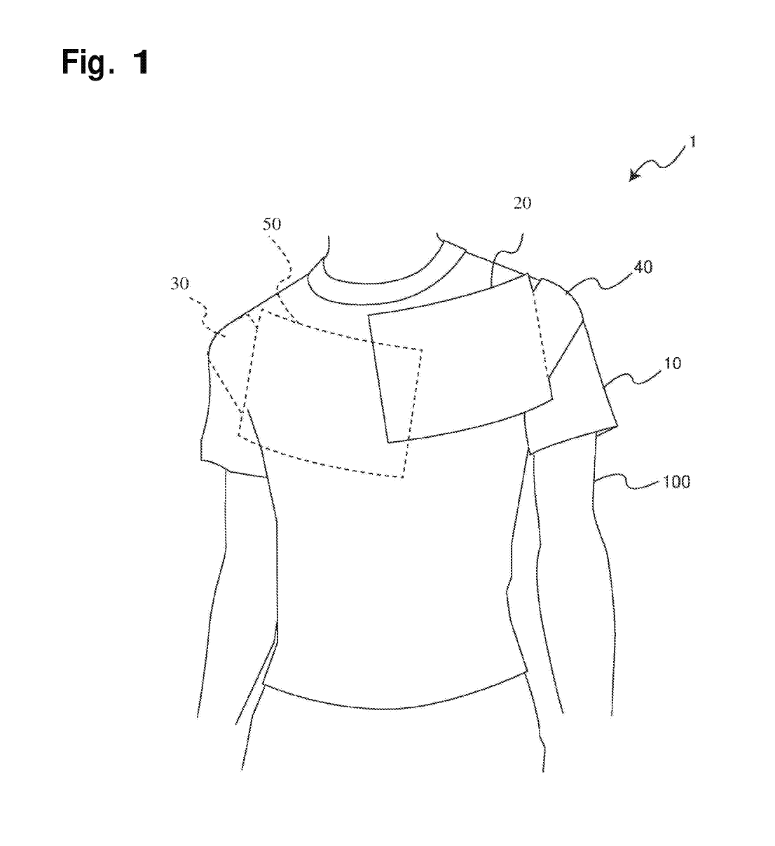

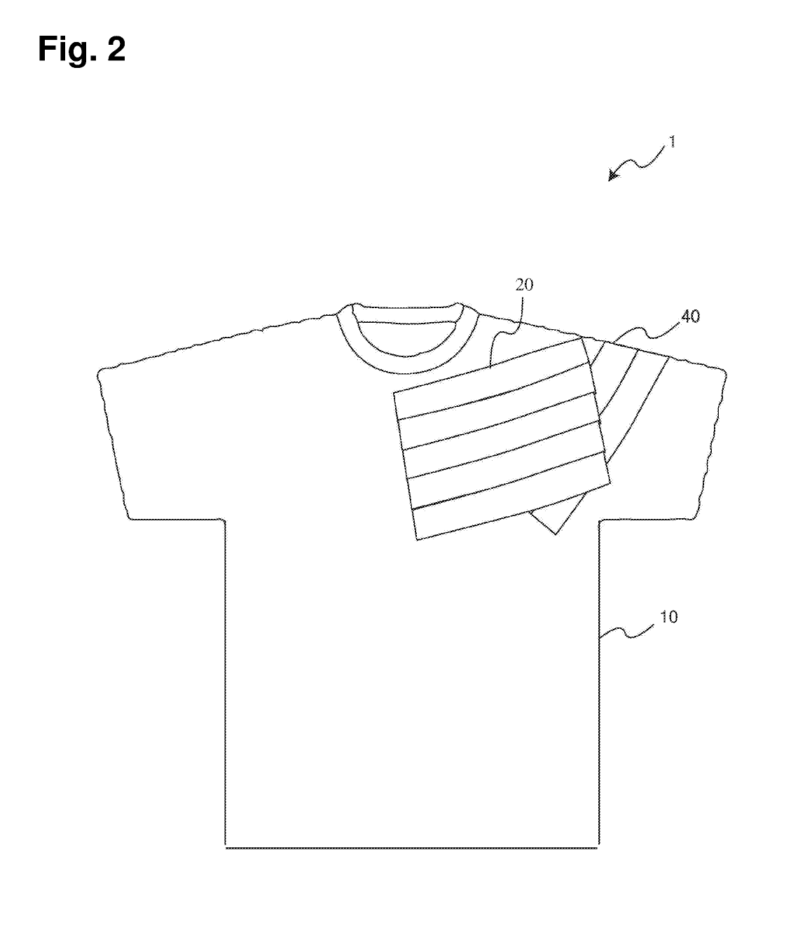

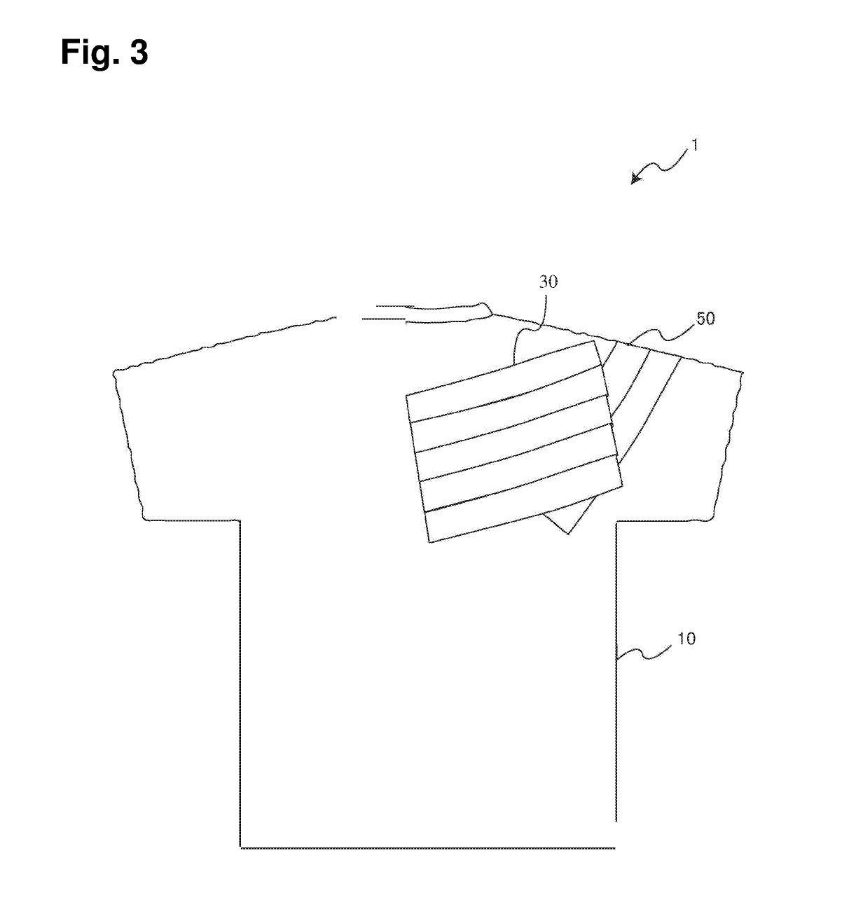

The structure of the shirt for correcting bone alignment 1 according to one embodiment of the present invention is described below with reference to the attached drawings. FIG. 1 is an in-use view of the shirt for correcting bone alignment 1 according to one embodiment of the present invention. FIG. 2 is a front view of the shirt for correcting bone alignment 1 according to this embodiment. FIG. 3 is a back view of the shirt for correcting bone alignment 1 according to this embodiment. The shirt for correcting bone alignment 1 corrects the alignment of the bone in the breast of the user 100 who is wearing. The shirt includes a shirt part 10, a first restraint part 20, a second restraint part 30, a first shoulder restraint part 40, and a second shoulder restraint part 50.

The shirt part 10 is formed from a stretch fabric. The shirt part 10 is in the form of a general T-shirt, etc.

The first restraint part 20 is formed on the right or the left side in the upper-half of the front body of the shirt part 10, which is less stretch than the fabric of the shirt part 10. The first restraint part 20 only has to be placed over an area from the collar of the shirt part 10 to the position corresponding to the sixth rib of the user who is wearing the shirt for correcting bone alignment 1. The first restraint part 20 is preferably formed to cover the area corresponding to from the first rib to the sixth rib of the user.

In the example shown in FIGS. 1 to 3, the first restraint part 20 is placed in the left side of the user. However, the first restraint part 20 can be placed in the right by wearing the shirt part 10 inside out. The first restraint part 20 is preferably formed to cover the area corresponding to the eighth rib from the back.

As shown in FIG. 3, the second restraint part 30 is formed on the different side from the first restraint part in the upper-half of the back body of the shirt part 10, which is less stretch than the fabric of the shirt part 10. When the user wears the shirt for correcting bone alignment 1, the second restraint part 30 is preferably formed to cover the area corresponding to one of the scapulas.

As shown in FIG. 2, the first shoulder restraint part 40 is placed over an area from one of the shoulder parts to the first restraint part 20 of the shirt part 10, which is less stretch than the fabric of the shirt part 10. The first shoulder restraint part 40 diagonally extends from one of the shoulder parts toward the center of the shirt part 10 and connects with the first restraint part 20.

As shown in FIG. 3, the second shoulder restraint part 50 is placed over an area from the other of the shoulder parts to the second restraint part 30 of the shirt part 10, which is less stretch than the fabric of the shirt part 10. The second shoulder restraint part 50 diagonally extends from the other of the shoulder parts toward the center of the shirt part 10 and connects with the second restraint part 30.

In the example shown in FIGS. 2 and 3, the first restraint part 20, the second restraint part 30, the first shoulder restraint part 40, and the second shoulder restraint part 50 are formed from a taping tape. These parts are preferably integrated with the shirt part 10 by using a material (e.g., resin or silicon) which is less stretch than the fabric of the shirt part 10.

The shirt for correcting bone alignment 1 according to this embodiment produces the following effects. When the user wears the shirt part 10, the first restraint part 20 and the second restraint part 30 which are less stretch than the fabric of the shirt part 10 are placed opposite from each other. The body of the user 100 is situated between the first restraint part 20 and the second restraint part 30. The first restraint part 20 restrains user's breast. Therefore, the shirt can correct the alignment of the bone in user's breast. Moreover, the portion other than the first restraint part 20 and the second restraint part 30 of the shirt part 10 are formed from a stretch fabric so that the shirt can reduce burden on user's body. As the result, the present invention can provide a shirt for correcting bone alignment that has a capability to correct the alignment of the sternum and the ribs and reduce burden on user's body.

Furthermore, if the shirt part 10 is worn inside out, the first restraint part 20 comes to the other side in the upper-half of the front body. The second restraint part 30 then comes to the different side from the first restraint part in the upper-half of the back body. Specifically, the first restraint part 20 can be horizontally reversed by wearing the shirt part 10 inside out. Therefore, the first restraint part 20 can be placed right or left depending on the alignment of the bone in the breast of the user 100.

The shirt for correcting bone alignment 1 further including a first shoulder restraint part 40 and a second shoulder restraint part 50 that the user is wearing can provide the same effect as when the user tapes the shoulder to reduce shoulder tension.

The present invention is not limited to the above-mentioned embodiment. The present invention includes variations and modifications without departing from the spirit and the scope of the present invention.

### REFERENCE SIGNS LIST

1 Shirt for correcting bone alignment

10 Shirt part

20 First restraint part

30 Second restraint part

40 First shoulder restraint part

50 Second shoulder restraint part

1. A shirt for correcting bone alignment that corrects the alignment of the bone in the breast of the user who is wearing, comprising:
a shirt part that is formed from a stretch fabric;
a first restraint part that is formed on the right or the left side in the upper-half of the front body of the shirt part, the first restraint part being less stretch than the fabric of the shirt part; and
a second restraint part that is formed on the different side from the first restraint part in the upper-half of the back body of the shirt part, the second restraint part being less stretch than the fabric of the shirt part.

2. The shirt for correcting bone alignment according to claim 1, further comprising:
a first shoulder restraint part that is placed over an area from one of the shoulder parts to the first restraint part of the shirt part, the first shoulder restraint part being less stretch than the fabric of the shirt part; and
a second shoulder restraint part that is placed over an area from the other of the shoulder parts to the second restraint part, the second shoulder restraint part being less stretch than the fabric of the shirt part.
